(19) United States
(12) Patent Application Publication
(10) Pub. No.: US 2010/0040547 A1
(43) Pub. Date: Feb. 18, 2010

(54) DYES AND PRECURSORS AND CONJUGATES THEREOF

(76) Inventor: John V. Frangioni, Wayland, MA (US)

Correspondence Address: FISH & RICHARDSON PC, P.O. BOX 1022, MINNEAPOLIS, MN, 55440-1022, US

(21) Appl. No.: 12/376,225

(22) PCT Filed: Aug. 3, 2007
(86) PCT No.: PCT/US07/75223

§ 371 Date: Oct. 15, 2009

Related U.S. Application Data
(60) Provisional application No. 60/835,407, filed on Aug. 3, 2006.
(60) Provisional application No. 60/835,344, filed on Aug. 3, 2006.

(51) Int. Cl. A61K 49/00; C07D 403/04; C07D 209/04; C07D 207/46

(52) U.S. Cl. 424/9.1; 548/455; 548/469; 548/427; 548/542

(57) ABSTRACT

Novel dyes, precursors to novel dyes, and conjugates of the novel dyes are disclosed, as well as methods of making and using the same.

### CROSS-REFERENCE TO RELATED APPLICATIONS

This application claims priority from U.S. Provisional Application Ser. No. 60/835,407, filed on Aug. 3, 2006, and U.S. Provisional Application Ser. No. 60/835,344, filed on Aug. 3, 2006, the contents of which are incorporated herein by reference in their entireties.

### TECHNICAL FIELD

This invention relates to dyes, and to precursors and conjugates thereof.

### BACKGROUND

Generally, cyanine dyes have a delocalized electron system that spans over many carbon atoms. FIG. 1 shows one such dye, 2-(2-[2-chloro-3-([1,3-dihydro-1,3,3-trimethyl-2H-indol-2-ylidene]ethylidene)-1-cyclohexen-1-yl]ethenyl)-1,3,3-trimethylindolium iodide, which is commonly known as IR-786 (1)A. The synthesis of some cyanine dyes is described in Little et al., U.S. Pat. No. 6,027,709; Lugade et al., U.S. Pat. No. 6,995,274, and U.S. Patent Application Publication No. 2006/0063247; Achilefu et al., U.S. Pat. No. 6,939,532; and Li et al., Synthesis and Characterization of Heptamethine Cyanine Dyes, Molecules, 2, 91-98 (1997).

Cyanine dyes, which often have an intense absorption and emission in the near-infrared (NIR) region, can be useful for biomedical fluorescence imaging because biological tissues are typically optically transparent in this region. Several studies on the use of NIR dyes, and dye-biomolecule conjugates have been published. For example, see Patonay et al., Near-Infrared Fluorogenic Labels: New Approach to an Old Problem, Analytical. Chemistry, 63:321A-327A (1991); Brinkley, A Brief Survey of Methods for Preparing Protein Conjugates with Dyes, Haptens, and Cross-Linking Reagents, Perspectives in Bioconjugate Chemistry, pp. 59-70, C. Meares (Ed), ACS Publication, Washington, D.C. (1993); Slavik, Fluorescent Probes in Cellular and Molecular Biology, CRC Press, Inc. (1994); Lee et al., U.S. Pat. No. 5,453,505; Hohenschuh et al., WO 98/48846; Turner et al., WO 98/22146; Kai et al., WO 96/17628; Snow et al., WO 98/48838; and Frangioni et al., IRDye78 Conjugates for Near-Infrared Fluorescence Imaging, Molecular Imaging, 1(4):354-364 (2002).

### SUMMARY

Generally, the new dyes and conjugates described herein have non-ionic solubilizing arms, which can effectively “shroud” the positive charge on the dye nucleus, reducing the overall effective charge of the molecule. This shrouding dramatically enhances the stability of the dyes and conjugates and their solubility in biological fluids. The enhanced solubility and stability of the new dyes and conjugates reduces non-specific background noise during surgery. In addition, the increased solubility enables the use of these new dyes in many biological applications.

As used herein, non-ionic solubilizing arms are neutral moieties, such as oligomers or polymers, that are capable of interacting strongly with, e.g., capable of forming hydrogen bonds with, water. Examples include polyethylene glycols (PEGs), polypropylene glycols, or copolymers of polyethylene oxide, and polypropylene oxide. For these specific examples, each oxygen atom on the molecular arm can interact strongly with a molecule of water.

More particularly, some of the new dyes herein include a positively charged nitrogen-containing dye core that includes a conjugated tri-, penta-, or heptamethine system. As used herein, “a heptamethine system” is an uninterrupted molecular fragment that includes seven methine groups (CH groups) and having a delocalized electron density, whereas tri-, and penta-methine moieties include three and five methine groups, respectively. The dye core has one or more non-ionic solubilizing molecular arms and, optionally, one or more functionalizable molecular arms bonded thereto. When present, the one or more functionalizable molecular arms include an amine-, alcohol-, or thiol-reactive carboxylic acid group, anhydride group, ester group, or isothiocyanate group. As used herein, “a functionalizable molecular arm” is a moiety that can be conjugated. For example, the molecular arm can be conjugated with a protein, or a carbohydrate. The dye core can include a single positive charge, or multiple charges. The tri-, penta- or heptamethine system can be substituted or unsubstituted.

Generally, the dyes have a high solubility in vitro, and in biological systems. For example, the one or more solubilizing molecular arms can be selected such that the dyes have a solubility in 10 mM HEPES solution (N-(2-hydroxyethyl)piperazine-N′-(2-ethanesulfonic acid)), pH 7.4, of greater than about 10 μM, e.g., greater than 25, 50, 75, 100, 125, 150, or even greater than 250 μM. If desired, the one or more solubilizing arms can also be functionalized with an amine-, alcohol-, or thiol-reactive carboxylic acid group, anhydride group, ester group, or isothiocyanate group. Generally, the dyes have an intense absorption and/or emission at a wavelength of from about 300 nm to 1000 nm, and thus emit in the green, yellow, orange, red, and near infrared portions of the spectrum. For example, the dyes can have a maximum excitation and/or a maximum emission, measured in 10 mM HEPES solution, pH 7.4, of from about 525 nm to about 875 nm, e.g., from about 550 nm to about 825 nm, or from about 550 nm to about 800 nm.

The one or more non-ionic solubilizing molecular arms can be, e.g., a polyethylene glycol, e.g., one terminated with a hydroxyl group or a alkoxy group. Conjugates can be formed by reacting the dyes with one or more molecular arms having suitable functionality, e.g., an amine-, alcohol-, or thiol-reactive carboxylic acid group, anhydride group, ester group, or isothiocyanate group. For example, such functionalized molecular arms can be conjugated with an amino-, hydroxyl-, or thiol-containing moiety, such as a small molecule peptide, protein, a polypeptide, or a carbohydrate.

In one aspect, the invention features compounds that include cations of Structure (I), which is shown below.

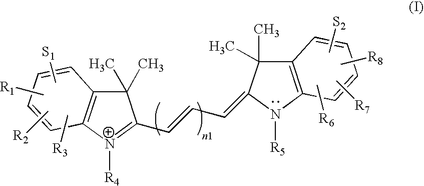

In such an aspect, S₁, and S₂ are each independently a non-ionic oligomeric or polymeric solubilizing moiety; n₁ is 1, 2 or 3; R₁, R₂, R₃, R₆, R₇, and R₈ are each independently H, F, Cl, Br, I, C1-C6 straight-chain or branched alkyl, C1-C6 straight-chain or branched alkoxy, an aromatic ring having up to 6 carbon atoms, optionally substituted with one or more F, Cl, Br or I. Any two or more of R₁, R₂ and R₃ and/or any two or more of R₆, R₇ and R₈ may be bonded together to define a ring that includes between 5 and 12 carbon atoms. The ring that includes between 5 and 12 carbon atoms can be optionally substituted with one or more F, Cl, Br, or I. R₄ and R₅ are each independently C1-C6 straight-chain or branched alkyl, an aromatic ring having up to 6 carbon atoms, optionally substituted with one or more F, Cl, Br or I, a non-ionic oligomeric or polymeric solubilizing moiety, or a moiety that includes at least one amine-, alcohol- or thiol-reactive carboxylic acid group, anhydride group, ester group, or isothiocyanate group. In some embodiments, the moiety that includes at least one amine-, alcohol-, or thiol-reactive carboxylic acid group, anhydride group, ester group, or isothiocyanate group also includes a solubilizing moiety.

In some embodiments, the compounds have cations which have a trimethine system represented by Structure (I′), a pentamethine system represented of Structure (I″) or a heptamethine system represented by Structure (I′″).

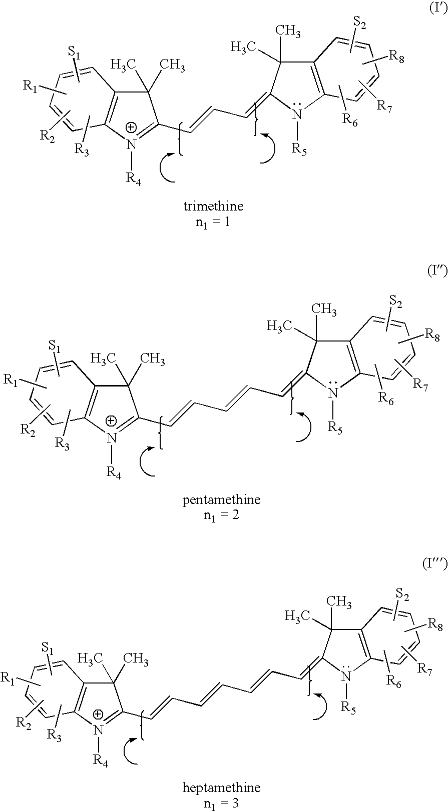

In another aspect, the invention features compounds of Structure (V), which is shown below.

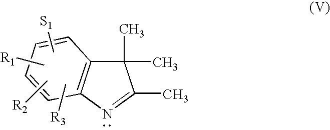

In such an aspect, S₁ is a non-ionic oligomeric or polymeric solubilizing moiety; and R₁, R₂, R₃ are each independently H, F, Cl, Br, I, C1-C6 straight-chain or branched alkyl, C1-C6 straight-chain or branched alkoxy, an aromatic ring having up to 6 carbon atoms, optionally substituted with one or more F, Cl, Br or I. Any two or more of R₁, R₂ and R₃ may be bonded together to define a ring that includes between 5 and 12 carbon atoms. The ring that includes 5-12 carbon atoms is optionally substituted with one or more F, Cl, Br, or I.

In another aspect, the invention features compounds that include cations of Structure (VI), which is shown below.

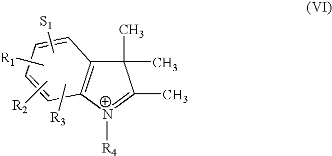

In such an aspect, S₁ is a non-ionic oligomeric or polymeric solubilizing moiety; and R₁, R₂, R₃ are each independently H, F, Cl, Br, I, C1-C6 straight-chain or branched alkyl, C1-C6 straight-chain or branched alkoxy, an aromatic ring having up to 6 carbon atoms, optionally substituted with one or more F, Cl, Br, or I. Any two or more of R₁, R₂ and R₃ may be bonded together to define a ring that includes between 5 and 12 carbon atoms. The ring that includes 5-12 carbon atoms is optionally substituted with one or more F, Cl, Br, or I. R₄ is independently C1-C6 straight-chain or branched alkyl, an aromatic ring having up to 6 carbon atoms, optionally substituted with one or more F, Cl, Br or I, a non-ionic oligomeric or polymeric solubilizing moiety, or a moiety that includes at least one amine, alcohol- or thiol-reactive carboxylic acid group, anhydride group, ester group, or isothiocyanate group.

In another aspect, the invention features compounds that include cations of Structure (VIII), which is shown below.

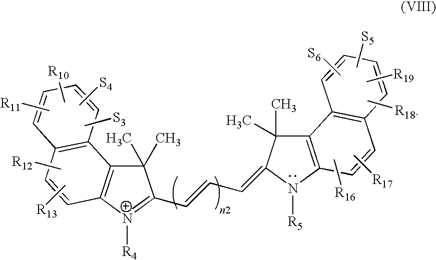

In such an aspect, S₃, S₄, S₅, and S₆ are each independently a non-ionic oligomeric or polymeric solubilizing moiety; n₂ is 1, 2 or 3; R₁₀, R₁₁, R₁₂, R₁₃, R₁₆, R₁₇, R₁₈, and R₁₉ are each independently H, F, Cl, Br, I, C1-C6 straight-chain or branched alkyl, C1-C6 straight-chain or branched alkoxy, an aromatic ring having up to 6 carbon atoms, optionally substituted with one or more F, Cl, Br or I. Any two or more of R₁₀, R₁₁, R₁₂, and R₁₃ and/or R₁₆, R₁₇, R₁₈, and R₁₉ may be bonded together to define a ring that includes between 5 and 1.2 carbon atoms. The ring that includes 5-12 carbon atoms is optionally substituted with one or more F, Cl, Br, or I. R₁₄ and R₁₅ are each independently C1-C6 straight-chain or branched alkyl, an aromatic ring having up to 6 carbon atoms, optionally substituted with one or more F, Cl, Br or I, a non-ionic oligomeric or polymeric solubilizing moiety, or a moiety that includes at least one amine-, alcohol- or thiol-reactive carboxylic acid group, anhydride group, ester group, or isothiocyanate group.

In some embodiments, the compounds have cations which have a trimethine system represented by Structure (VIII′), a pentamethine system represented of Structure (VIII″) or a heptamethine system represented by Structure (VIII′″).

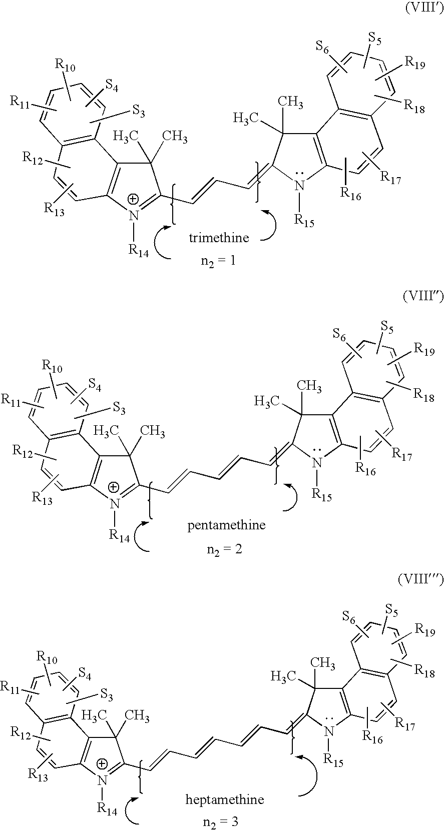

In another aspect, the invention features compounds of Structure (XII), which is shown below.

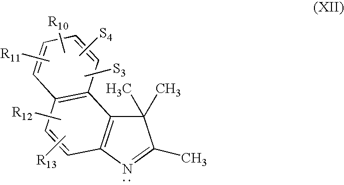

In such an aspect, S₃ and S₄ are each independently a non-ionic oligomeric or polymeric solubilizing moiety; and R₁₀, R₁₁, R₁₂, and R₁₃ are each independently H, F, Cl, Br, I, C1-C6 straight-chain or branched alkyl, C1-C6 straight-chain or branched alkoxy, an aromatic ring having up to 6 carbon atoms, optionally substituted with one or more F, Cl, Br, or I. Any two or more of R₁₀, R₁₁, R₁₂, and R₁₃ may be bonded together to define a ring that includes between 5 and 12 carbon atoms. The ring that includes 5-12 carbon atoms is optionally substituted with one or more F, Cl, Br, or I.

In another aspect, the invention features compounds that include cations of Structure (XIII), which is shown below.

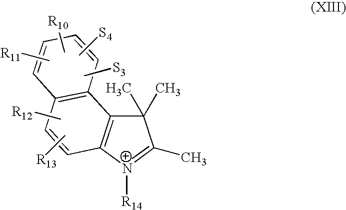

In such an aspect, S₃ and S₄ are each independently a non-ionic oligomeric or polymeric solubilizing moiety; R₁₀, R₁₁, R₁₂, and R₁₃ are each independently H, F, Cl, Br, I, C1-C6 straight-chain or branched alkyl, C1-C6 straight-chain or branched alkoxy, or an aromatic ring having up to 6 carbon atoms, optionally substituted with one or more F, Cl, Br, or I. Any two or more of R₁₀, R₁₁, R₁₂, and R₁₃ may be bonded together to define a ring that includes between 5 and 12 carbon atoms. The ring that includes 5-12 carbon atoms is optionally substituted with one or more F, Cl, Br, or I. R₁₄ is C1-C6 straight-chain or branched alkyl, an aromatic ring having up to 6 carbon atoms, optionally substituted with one or more F, Cl, Br or I, a non-ionic oligomeric or polymeric solubilizing moiety, or a moiety that includes at least one amine-, alcohol- or thiol-reactive carboxylic acid group, anhydride group, ester group, or isothiocyanate group.

Aspects and/or embodiments of the invention can have any one of, or combinations of, any of the following advantages. The dye precursors, dyes, and conjugates have a high solubility in aqueous solutions, and biological fluids and tissues. The dyes and conjugates have non-ionic solubilizing arms, which can effectively “shroud” the positive charge on the nitrogen atoms, reducing the overall effective charge of the molecule. Reducing the overall effective charge minimizes non-specific background noise during imaging. The dyes and conjugates can be used for real time surgical guidance for identifying tumors and other abnormal tissues. The dyes and conjugates have a high in vivo stability. The dyes can be easily conjugated with targeting molecules, such as those that contain an amino, thiol, and/or hydroxyl functionality. The dyes and conjugates retain high fluorescent yield at about 800 nm, which is often optimal for in vivo imaging. Solubilizing arms on the dyes and conjugates have a length that can be adjusted to optimize biodistribution and clearance. The solubilizing arms of the dyes and conjugates can reduce non-specific background binding in vivo. The dyes and conjugates can have a low overall toxicity.

For the purposes of this disclosure, 10 mM HEPES solution, pH 7.4, is a pH adjusted, 10 mM solution of N-(2-hydroxyethyl)piperazine-N′-(2-ethanesulfonic acid).

For mixtures of materials, such as mixtures of monomeric compounds or polymeric compounds that have a molecular weight distribution, solubility is the average solubility of the dye core.

An “oligomer” as used herein, is a relatively low molecular weight polymer having between about 4 and about 25 repeat units.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference herein in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is a resonance structure for 2-(2-[2-chloro-3-([1,3-dihydro-1,3,3-trimethyl-2H-indol-2-ylidene]ethylidene)-1-cyclohexen-1-yl]ethenyl)-1,3,3-trimethylindolium iodide (IR-786, (1)A).

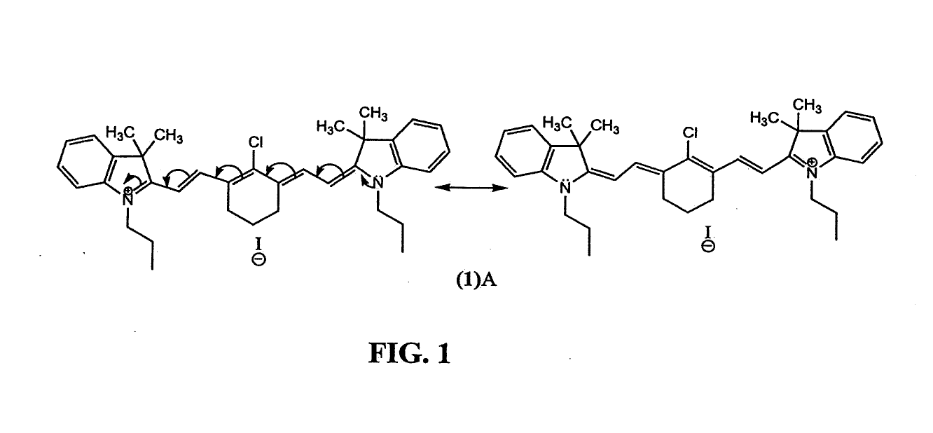

FIG. 2A is a generalized reaction scheme, illustrating attachment of solubilizing arms onto functionalized anilines.

FIG. 2B is a representation of eight structures of specific functionalized anilines and corresponding anilines having attached solubilizing arms.

FIG. 3 is a generalized reaction scheme, illustrating preparation of diazonium salts (not shown) corresponding to the anilines of FIG. 2 having the solubilizing arms, and then reduction of the diazonium salts to produce the corresponding hydrazines.

FIG. 4 is a generalized reaction scheme, illustrating cyclization of the hydrazines of FIG. 3, utilizing methyl isopropyl ketone and the Fischer indole reaction.

FIG. 5 is a generalized reaction scheme, illustrating quaternization of the cyclized products of FIG. 4.

FIG. 6 is a representation of four specific structures that can be used to quaternize the cyclized products of FIG. 5.

FIG. 7 is a generalized reaction scheme, illustrating coupling of the quaternized products of FIG. 5.

FIG. 8 is a generalized reaction scheme, illustrating preparation of other diazonium salts (not shown) from the shown anilines having solubilizing arms, and then reduction of the diazonium salts to produce the corresponding hydrazines.

FIG. 9 is a generalized reaction scheme, illustrating cyclization of the hydrazines of FIG. 8, utilizing methyl isopropyl ketone and the Fischer indole reaction.

FIG. 10 is a generalized reaction scheme, illustrating coupling of quaternized products corresponding to the cyclized products of FIG. 9.

FIG. 11 is a generalized reaction scheme showing the preparation of a conjugate from a dye and a hydroxyl-containing moiety, e.g., a carbohydrate.

FIG. 12 is a generalized reaction scheme, illustrating the preparation of a conjugate from a dye and an amino-containing moiety, e.g., a protein.

### DETAILED DESCRIPTION

Novel dyes are provided that include non-ionic solubilizing moieties, such as polyethylene glycols (PEGs). In many embodiments, the dyes can be conjugated, e.g., by reacting the dyes with a small molecule peptide, a protein or a carbohydrate, to provide imaging agents that can bind selectively to certain tissues, e.g., abnormal tissues, allowing for their imaging. For example, dyes and conjugates can be used for real time surgical guidance for identifying tumors, and other abnormal tissues.

### Dyes

Some dyes are provided that include cations represented by Structure (I), which is shown below.

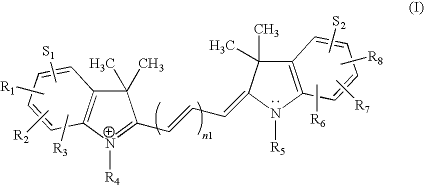

In dyes that include cations of Structure (I), S₁, and S₂ are each independently a non-ionic oligomeric or polymeric solubilizing moiety.

For example, each non-ionic oligomeric or polymeric solubilizing moiety can be a polyethylene glycol, a polypropylene glycol, a copolymer of polyethylene oxide and propylene oxide, a carbohydrate, a detran, or a polyacrylamide. Each solubilizing moiety on a particular molecule can be the same or different.

Each solubilizing moiety can be attached to the dye nucleus by any desired mode. For example, a moiety can be attached to the dye nucleus by bonding a terminal end (e.g., that contains a hydroxyl group), or a non-terminal end of the moiety to the dye nucleus. The point of attachment of the dye nucleus to the solubilizing moiety can be, e.g., a carbon-carbon bond, a carbon-oxygen, or a nitrogen-carbon bond. The attachment group for the solubilizing moiety to the dye nucleus can be, e.g., an ester group, a carbonate group, a ether group, a sulfide group, an amino group, an alkylene group, an amide group, a carbonyl group, or a phosphate group.

Specific examples of solubilizing groups are polyethylene glycols, such as —OC(═O)O(CH₂CH₂O)ₙH, —OC(═O)O(CH₂CH₂O)ₙCH₃, —O(CH₂CH₂O)ₙCH₃, —S(CH₂CH₂O)ₙCH₃, n being an integer between about 10 and about 250; and dextrans, such as —OC(═O)O(dextran).

Each solubilizing moiety can have an absolute molecular weight of from about 500 amu to about 100,000 amu, e.g., from about 1,000 amu to about 50,000 amu, or from about 1,500 to about 25,000 amu.

In some embodiments, S₁, and S₂ are selected such that the dyes that include the cations of Structure (I) have a solubility in 10 mM HEPES solution, pH 7.4, of greater than about 10 μM, e.g., greater than 25, 50, 75, 100, 125, 150, 200, or even greater than 250 μM. Solubility can be determined photometrically at 25° C. by setting up a calibration curve using a base dye core; saturating a 10 mM HEPES solution, pH 7.4, with the test compound or mixture, and then determining where on the calibration curve the test compound or mixture falls.

In some specific embodiments, S₁ and S₂ of compounds of Structure (I), are each independently of the form R₉(α)_{φ}, wherein φ is 0 or 1, α is O, S, CH₂, CH₂O, CO₂, or NR′ in which R′ is H or C1-C6 straight-chain or branched alkyl. In such instances, R₉ is of the form (CH₂CH₂O)ₙ₃R″ in which R″ is H or C1-C6 straight-chain or branched alkyl, n₃ being an integer from 4 to 2,500. In such pegylated dyes, when φ takes on the value of 0, α is not present, and R₉ is bonded directly to the indicated benzene ring. In particular embodiments, n₃ is between 6 and 2,000, e.g., between 10 and 1,000 or between 10 and 750.

In some specific embodiments, the PEG chain length and the PEG end group are selected such that the dyes that include the cations of Structure (I) have a solubility in 10 mM HEPES solution, pH 7.4, of greater than about 10 μM, e.g., greater than 25, 50, 75, 100, 125, 150, 200, or even greater than 250 μM.

In some specific embodiments, α is O or S and S₁, and S₂ are each independently of the form (CH₂CH₂O)ₙ₃R″, in which R″ is H and n₃ is an integer from 10 to 1,000.

In the dyes that include cations of Structure (I), n₁ is 1, 2 or 3, corresponding respectively to a compound having a trimethine spacer bridging nitrogen-containing heterocyclic rings, compounds having a pentamethine spacer and compounds having a heptamethine spacer bridging nitrogen-containing heterocyclic rings. In particular, compounds having a trimethine spacer, a pentamethine spacer and a heptamethine spacer are represented by Structures (I′), (I″) and (I′″), respectively (shown below).

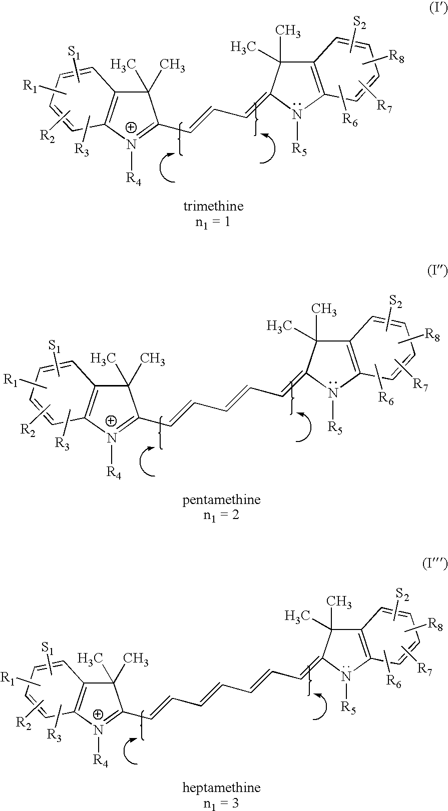

In dyes that include cations of Structure (I), R₁, R₂, R₃, R₆, R₇, and R₈ are each independently H, F, Cl, Br, I, C1-C6 straight-chain or branched alkyl, C1-C6 straight-chain or branched alkoxy, an aromatic ring having up to 6 carbon atoms, optionally substituted with one or more F, Cl, Br or I, or any two or more of R₁, R₂ and R₃ and/or R₆, R₇ and R₈ may be bonded together to define a ring that includes between 5 and 12 carbon atoms. The 5-12 carbon ring can be optionally substituted with one or more F, Cl, Br, or I. The ring that includes between 5 and 12 carbon atoms can a carbocyclic ring (e.g., a carbocyclic aromatic ring such as a phenyl group or a substituted phenyl group), or a heterocyclic ring (e.g., a heterocyclic aromatic ring, such as one containing nitrogen, oxygen sulfur or phosphorus).

In some specific embodiments, R₁, R₂, R₃, R₆, R₇, and R₈ are each H.

In dyes that include cations of Structure (I), R₄ and R₅ are each independently C1-C6 straight-chain or branched alkyl, an aromatic ring having up to 6 carbon atoms, optionally substituted with one or more F, Cl, Br, or I, a non-ionic oligomeric or polymeric solubilizing moiety, or a moiety that includes at least one amine-, alcohol- or thiol-reactive carboxylic acid group, anhydride group, ester group, or isothiocyanate group.

The moiety that includes at least one amine-, alcohol- or thiol-reactive carboxylic acid group, anhydride group, ester group, or isothiocyanate group allows the dyes to be conjugated with another compound that includes an amino group (e.g., a small molecule peptide, or a protein), an alcohol group (e.g., a carbohydrate), or a thiol group; or a non-ionic oligomeric or polymeric solubilizing moiety. If desired, e.g., to improve solubility or biocompatibility, the moiety that includes at least one e.g., to improve solubility or biocompatibility, the moiety that includes at least one amine-, alcohol- or thiol-reactive carboxylic acid group, anhydride group, ester group, or isothiocyanate group can include any of the solubilizing moieties discussed herein. For example, the solubilizing group can act as a spacer between the dye nucleus and the amine-, alcohol- or thiol-reactive carboxylic acid group, anhydride group, ester group, or isothiocyanate group.

In some specific embodiments, R₄ is

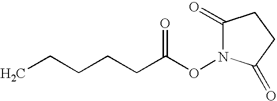

and R₅ is C1-C6 straight or branched alkyl, e.g., methyl, ethyl, isopropyl, or n-pentyl.

In some specific embodiments, both R₄ and R₅ include a moiety that includes at least one amine-, alcohol- or thiol-reactive carboxylic acid group, anhydride group, ester group, or isothiocyanate group, allowing both R₄ and R₅ to be conjugated.

Examples of C1-C6 straight-chain or branched alkyl groups include methyl, ethyl, n-propyl, isopropyl, n-pentyl, isopentyl and neopentyl. Examples of C1-C6 straight-chain or branched alkoxy groups include methoxy, ethoxy, n-propoxy, isopropoxy, n-pentoxy, isopentoxy and neopentoxy.

Examples of aromatic ring systems having up to 6 carbon atoms, optionally substituted with one or more F, Cl, Br, or I, include phenyl groups or substituted phenyl groups (e.g., an attached benzene ring having 1,2-dichloro substitution or 1-chloro-4-fluoro substitution), and heterocyclic aromatic groups or substituted heterocyclic aromatic groups, such as furan, thiophene, imidazole, pyrazole, oxazole, pyridine, and their substituted derivatives.

Any of the compounds of Structure (I) can have a counterion (A⁻) that is inorganic, such as F⁻, Cl⁻, Br⁻, I⁻, ClO₄⁻, or a counterion that is organic, such as CH₃COO⁻, formate ion, or citrate ion.

Some dyes are provided that include cations represented by Structure (VIII), which is shown below.

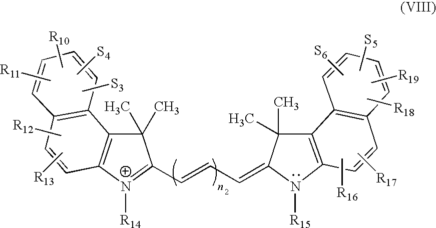

In dyes that include cations of Structure (VIII), S₃, S₄, S₅ and S₆ are each independently a non-ionic oligomeric or polymeric solubilizing moiety.

In some embodiments, S₃, S₄, S₅ and S₆ are selected such that the dyes that include the cations of Structure (VIII) have a solubility in 10 mM HEPES (N-(2-hydroxyethyl)piperazine-N′-(2-ethanesulfonic acid)) solution, pH 7.4, of greater than about 10 μM, e.g., greater than 25, 50, 75, 100, 125, 150, 200, or even greater than 250 μM.

Each solubilizing moiety can be any of those discussed above in reference to Structure (I). For example, each non-ionic oligomeric or polymeric solubilizing moiety can be a polyethylene glycol, which is attached to the dye nucleus by any of the modes discussed above. Each solubilizing moiety can have an absolute molecular weight as discussed above. For example, the absolute molecular weight of each solubilizing moiety can be from about 1,000 amu to about 50,000 amu.

In some specific embodiments, S₃, S₄, S₅ and S₆ of compounds of Structure (VIII) are each independently of the form R₉(α)_{φ}, wherein φ is 0 or 1, α is O, S, CH₂, CH₂O, CO₂, or NR′ in which R′ is H or C1-C6 straight-chain or branched alkyl. In such instances, R₉ is of the form (CH₂CH₂O)ₙ₃R″ in which R″ is H or C1-C6 straight-chain or branched alkyl, n₃ being an integer from 4 to 2,500.

In the dyes that include cations of Structure (VIII), n₂ is 1, 2, or 3, corresponding respectively to a compounds having a trimethine spacer bridging nitrogen-containing heterocyclic rings, compounds having a pentamethine spacer, and compounds having a heptamethine spacer bridging nitrogen-containing heterocyclic rings. In particular, compounds having a trimethine spacer, a pentamethine spacer, and a heptamethine spacer are represented by Structures (VIII′), (VIII″) and (VIII′″) respectively (shown below).

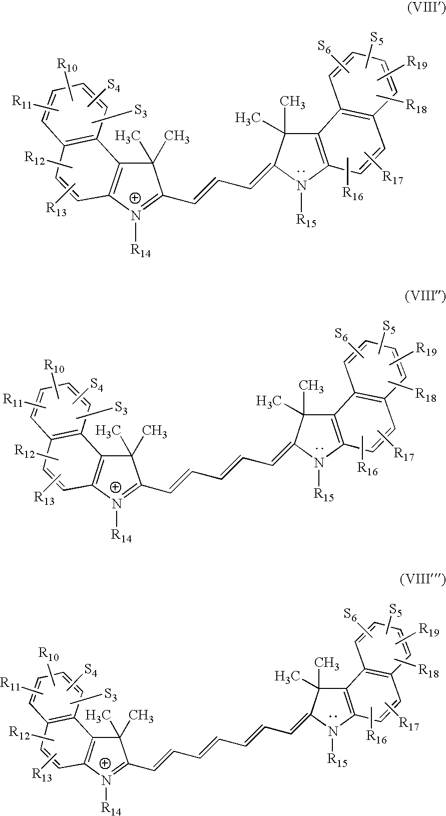

In dyes that include cations of Structure (VIII), R₁₀, R₁₁, R₁₂, R₁₃, R₁₆, R₁₇, R₁₈, and R₁₉ are each independently H, F, Cl, Br, I, C1-C6 straight-chain or branched alkyl, C1-C6 straight-chain or branched alkoxy, an aromatic ring having up to 6 carbon atoms, optionally substituted with one or more F, Cl, Br or I, or any two or more of R₁₀, R₁₁, R₁₂, and R₁₃ and/or R₁₆, R₁₇, R₁₈, and R₁₉ may be bonded together to define a ring that includes between 5 and 12 carbon atoms. The 5 to 12 carbon atom ring can be optionally substituted with one or more F, Cl, Br, or I.

In dyes that include cations of Structure (VIII), R₁₄ and R₁₅ are each independently C1-C6 straight-chain or branched alkyl, an aromatic ring having up to 6 carbon atoms, optionally substituted with one or more F, Cl, Br or I, a non-ionic oligomeric or polymeric solubilizing moiety, or a moiety that includes at least one amine-, alcohol- or thiol-reactive carboxylic acid group, anhydride group, ester group, or isothiocyanate group.

In some specific embodiments, both R₁₄ and R₁₅ include a moiety that includes at least one amine-, alcohol- or thiol-reactive carboxylic acid group, anhydride group, ester group, or isothiocyanate group, allowing both R₁₄ and R₁₅ to be conjugated.

Examples of C1-C6 straight chain or branched alkyl, C1-C6 straight-chain or branched alkoxy, aromatic rings having up to 6 carbon atoms, and the moiety that includes at least one amine-, alcohol-, or thiol-reactive carboxylic acid group, anhydride group, ester group, or isothiocyanate group have been discussed above. If desired, e.g., to improve solubility or biocompatibility, the moiety that includes at least one amine-, alcohol- or thiol-reactive carboxylic acid group, anhydride group, ester group, or isothiocyanate group can include any of the solubilizing moieties discussed above. For example, the solubilizing group can act as a spacer between the dye nucleus and the amine-, alcohol- or thiol-reactive carboxylic acid group, anhydride group, ester group, or isothiocyanate group. When any two or more of R₁₀, R₁₁, R₁₂, and R₁₃ and/or R₁₆, R₁₇, R₁₈, and R₁₉ are bonded together to define a ring that includes between 5 and 12 carbon atoms (which is optionally, substituted with one or more F, Cl, Br, or I), the ring can be carbocyclic or heterocyclic, as discussed above in reference to compounds of Structure (I).

Any of the compounds of Structure (VIII) can have a counterion (A⁻) that is inorganic, such as F⁻, Cl⁻, Br⁻, I⁻, ClO₄⁻, or a counterion that is organic, such as CH₃COO⁻, formate ion, or citrate ion.

### Absorption and Emission Properties of the Dyes

Generally, the dyes intensely absorb and emit light in the visible, and infrared region of the electromagnetic spectrum, e.g., they can emit green, yellow, orange, red light, or near-infrared (“NIR”) light.

In some embodiments, the dyes emit and/or absorb radiation having a wavelength from about 300 nm to about 1000 nm, e.g., from about 400 nm to about 900 nm, or from about 450 nm to about 850 nm.

In some embodiments, the dyes have a maximum excitation and/or a maximum emission, measured in 10 mM HEPES solution, pH 7.4, of from about 525 nm to about 875 nm, e.g., from about 550 nm to about 825 nm, or from about 550 nm to about 800 nm.

### Methods of Preparing the Dyes

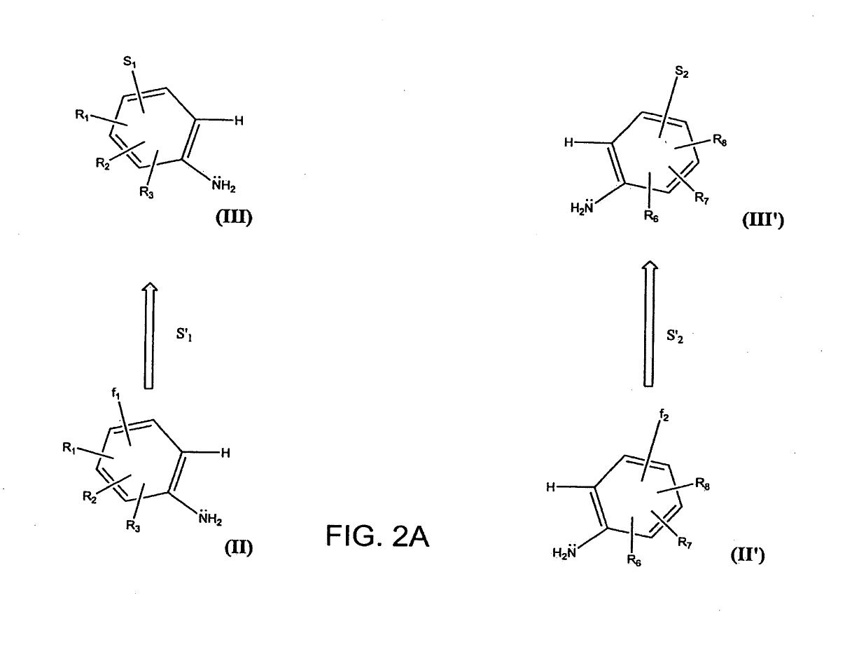

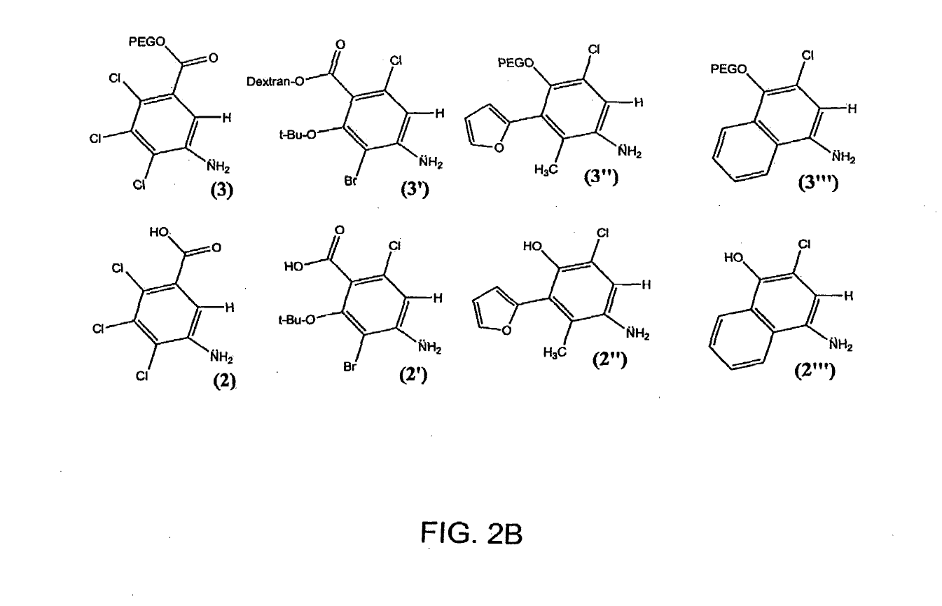

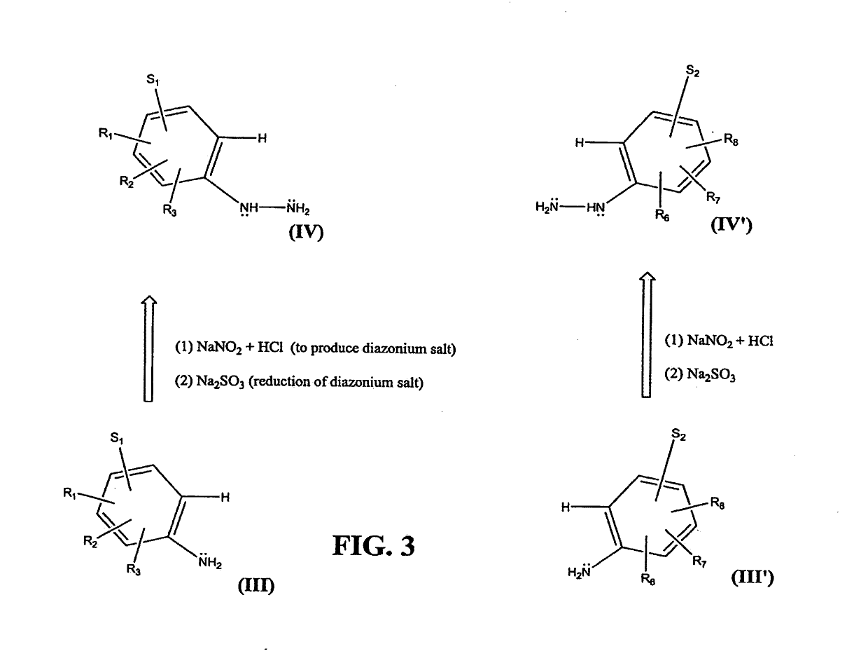

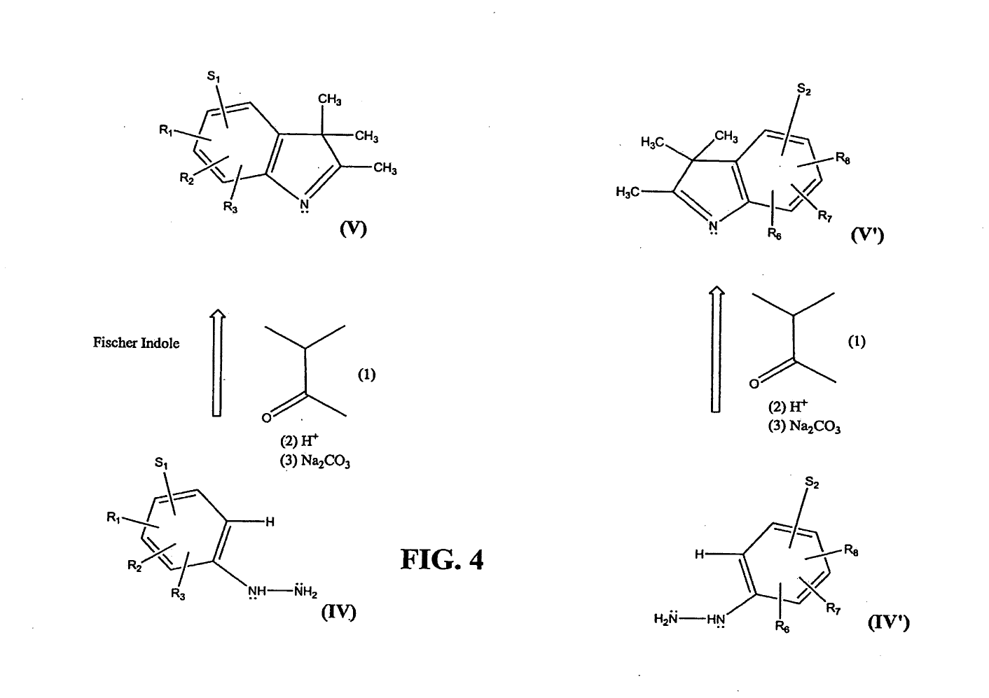

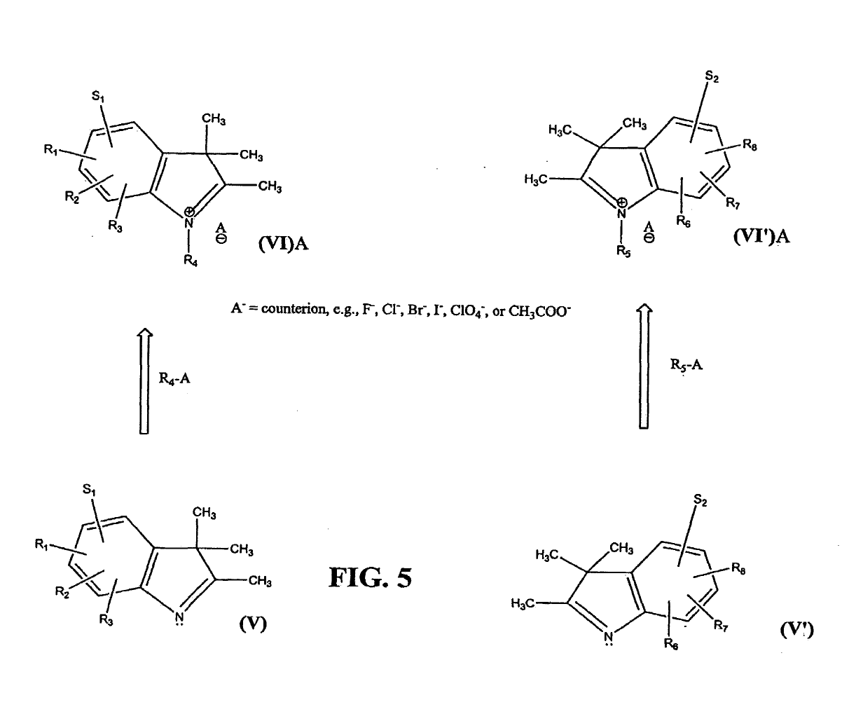

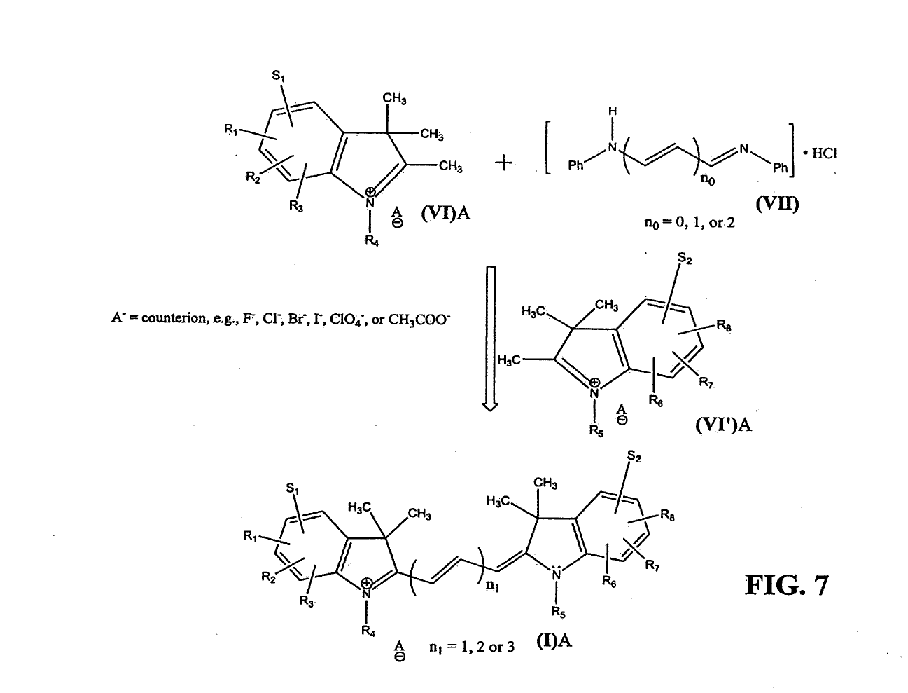

As an overview, FIGS. 2A-7 show that dyes of Structure (I)A (FIG. 7), which include cations of Structure (I), can be prepared by first attaching solubilizing arms onto the desired functionalized anilines (FIG. 2A). The resulting anilines having the solubilizing arms are converted to the corresponding hydrazines (FIG. 3), and then the hydrazines are cyclized using methyl isopropyl ketone and the Fischer Indole reaction (FIG. 4). The heterocycles thus formed are then quaternized by attachment of groups or arms, e.g., solubilizing arms, to the nitrogen atom of each heterocycle (FIG. 5). Finally, the quaternized heterocycles are coupled using the desired formamidine or dienylidene (VII) (FIG. 7). This particular synthetic scheme is described in a little more detail below.

Referring particularly to FIG. 2A, functionalized anilines of Structures (II) and (II′) are reacted with S′₁ or S′₂, respectively, converting each respective functional group f₁ or f₂ to solubilizing arms S₁ or S₂, to generate anilines of Structures (III) and (III′). Functional groups f₁ and f₂ can be, e.g., a carboxylic acid group (or an ester thereof), or a phenolic oxide group (formed by deprotonating a phenolic hydroxyl group), and S′₁ or S′₂ can be, e.g., α,ω-di-hydroxy polyethylene oxide, dextran, or ethylene oxide. R₁, R₂, and R₃ can be any of the groups described above in reference to Structure (I) above. Specific examples of the functionalized anilines prior to attaching solubilizing arms include those shown in FIG. 2A (i.e., compounds 2, 2′, 2″ and 2′″). Specific examples of anilines having attached solubilizing arms are also shown in FIG. 2B (i.e., compound 3, 3′, 3″ and 3′″).

Referring particularly to FIG. 3, anilines having solubilizing arms represented by Structures (III) and (III′) are each reacted with, e.g., NaNO₂, which produces each respective diazonium salt (not shown). Reduction of each diazonium salt, e.g., using Na₂SO₃, generates the corresponding hydrazine, represented by Structure (IV) or (IV′).

Referring particularly to FIG. 4, hydrazines of Structures (IV) and (IV′) are each cyclized using methyl isopropyl ketone and the Fischer Indole reaction, generating the corresponding heterocycles, represented by Structures (V) and (V′).

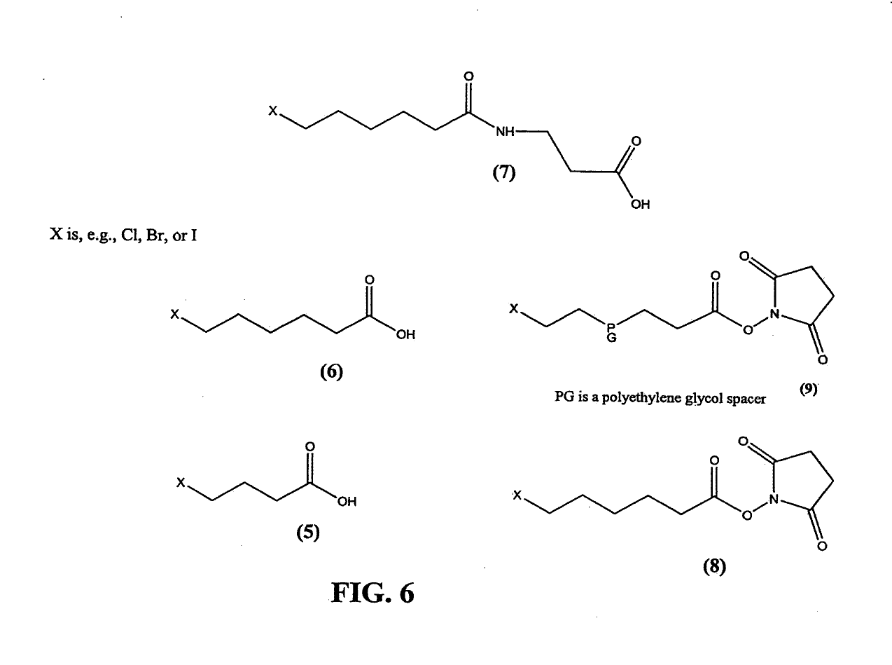

Referring particularly to FIG. 5, neutral heterocycles of Structures (V) and (V′) are quaternized using, e.g., R₄A and R₅A, respectively, generating quaternized heterocyclic compounds of Structures (VI)A and (VI′)A, A being the counterion (e.g., Cl⁻, Br⁻, or I⁻). If desired, R₄A and R₅A can be, e.g., a solubilizing moiety that includes a good leaving group, such as a halogen. In particular embodiments, R₄A and/or R₅A are polyethylene glycols that have a terminal bromide and terminal amine-, alcohol- or thiol-reactive carboxylic acid group, anhydride group, ester group, or isothiocyanate group. Specific examples of R₄A and R₅A are shown in FIG. 6 (i.e., compounds 5, 6, 7, 8, and 9)

Referring particularly to FIG. 7, quaternized heterocyclic compounds of Structures (VI)A and (VI′)A are coupled by first reacting one of (VI)A or (VI′)A with (VII), and then reacting the reaction product with (VI)A or (VI′)A.

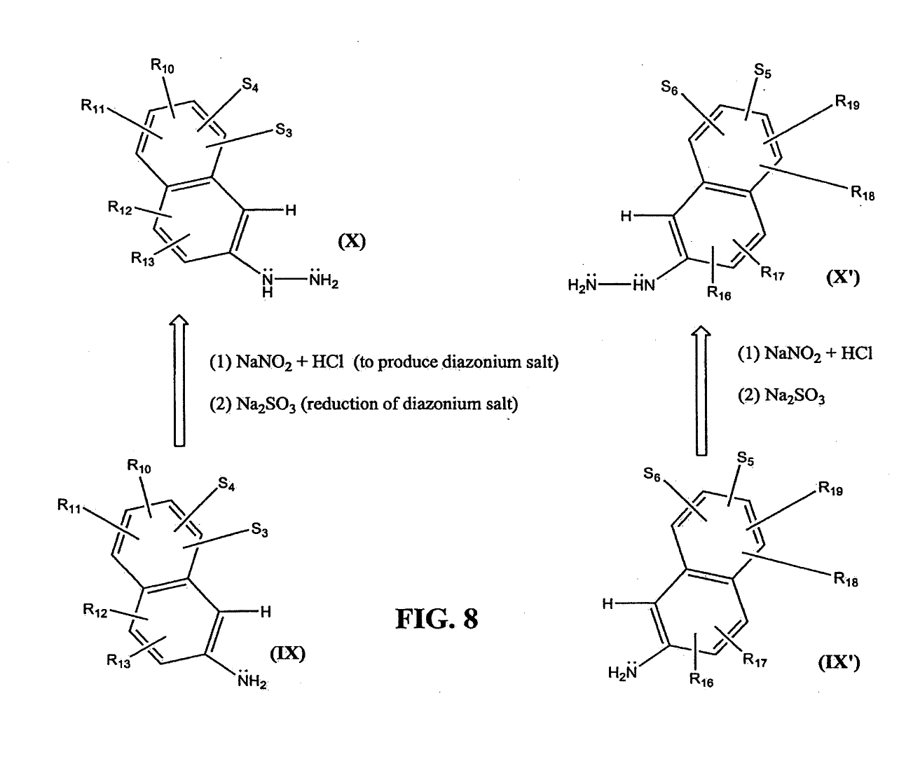

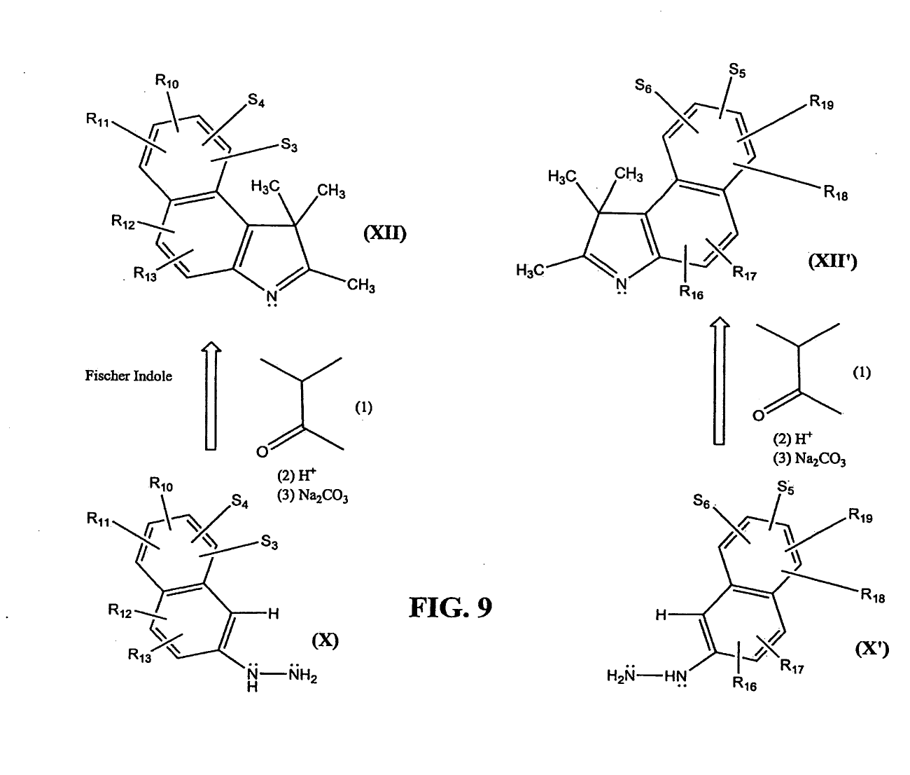

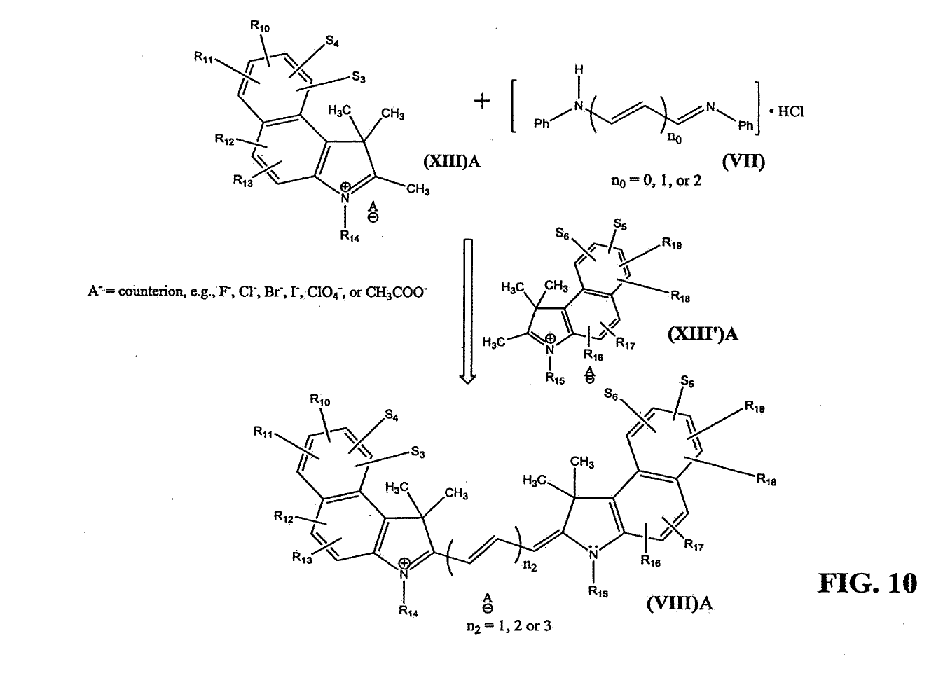

As an overview, FIGS. 8-10 show that dyes of Structure (VIII)A (FIG. 10), which include cations of Structure (VIII), can be prepared by first attaching solubilizing arms onto the desired functionalized anilines (not shown, but analogous to that shown in FIG. 2). The resulting anilines having the solubilizing arms are converted to the corresponding hydrazines (FIG. 8), and then the hydrazines are cyclized using methyl isopropyl ketone and the Fischer Indole reaction (FIG. 9). The heterocycles thus formed are then quaternized (not shown, but analogous to that shown in FIG. 5). Finally, the quaternized heterocycles are coupled using the desired formamidine or dienylidene (VII). (FIG. 10). This particular synthetic scheme is described in a little more detail below.

Functionalized anilines are reacted with S′₃ and S′₄, and S′₅ and S′₆, respectively, converting each respective functional group f₃ and f₄, and f₅ and f₆ to solubilizing arms S₃ and S₄ or S₅ and S₆, to generate anilines of Structures (IX) and (IX′). S₃-S₆ and R₁₀-R₁₉, can be any of the groups described above in reference to Structure (VIII) above.

Referring particularly to FIG. 8, anilines having solubilizing arms represented by Structures (IX) and (IX′) are each reacted with, e.g., NaNO₂, which produces each respective diazonium salt (not shown). Reduction of each diazonium salt, e.g., using Na₂SO₃, generates the corresponding hydrazine, represented by Structure (X) or (X′).

Referring particularly to FIG. 9, hydrazines of Structures (X) and (X′) are each cyclized using methyl isopropyl ketone and the Fischer Indole reaction, generating the corresponding heterocycles, represented by Structures (XII) and (XII′).

Neutral heterocycles of Structures (XII) and (XII′) are then each quaternized using, e.g., R₁₄A and R₁₅A, respectively, generating quaternized heterocyclic compounds of Structures (XIII)A and (XIII′)A, A being the counterion (e.g., Cl⁻, Br⁻, or I⁻). If desired, R₁₄A and R₁₅A can be, e.g., a solubilizing moiety that includes a good leaving group, such as a halogen. In particular embodiments, R₁₄A and/or R₁₅A are polyethylene glycols that have a terminal bromide and terminal amine-, alcohol- or thiol-reactive carboxylic acid group, anhydride group, ester group, or isothiocyanate group.

Referring particularly to FIG. 10, quaternized heterocyclic compounds of Structures (XIII)A and (XIII′)A are coupled by first reacting one of (XIII)A or (XIII′)A with (VII), and then reacting the reaction product with (XIII)A or (XIII′)A.

When desired and/or necessitated to effect any chemical transformation, any of the functional groups in any of the synthetic schemes shown herein can be protected by protecting groups, which can be removed in a later step to produce the desired compound.

Other synthetic schemes that can be applied to making dyes are described in Frangioni et al., U.S. Provisional Patent Application Ser. No. 60/835,344, filed Aug. 3, 2006.

### Dye Conjugates

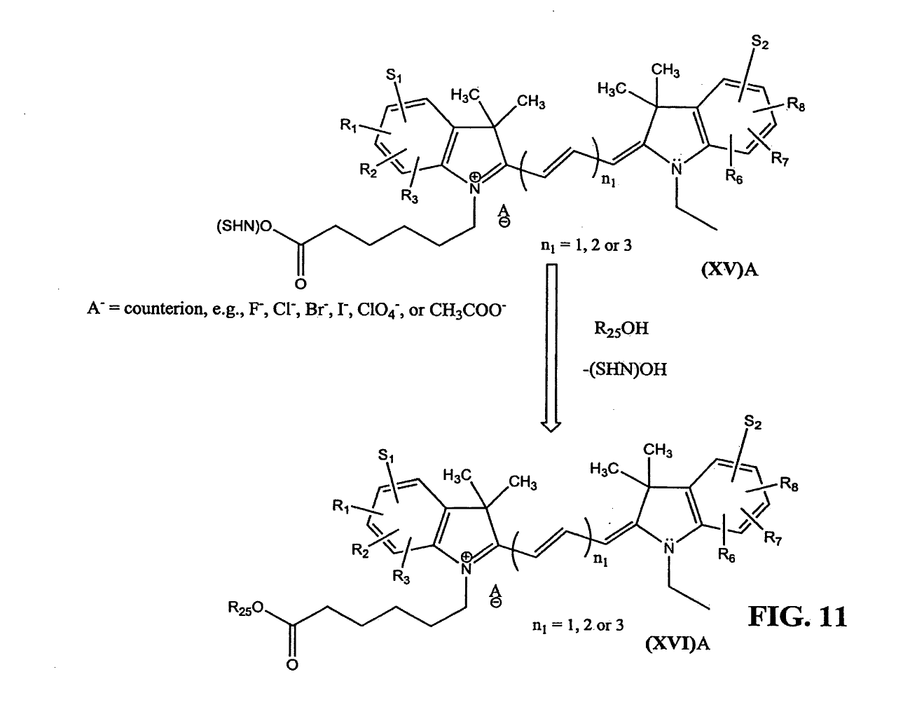

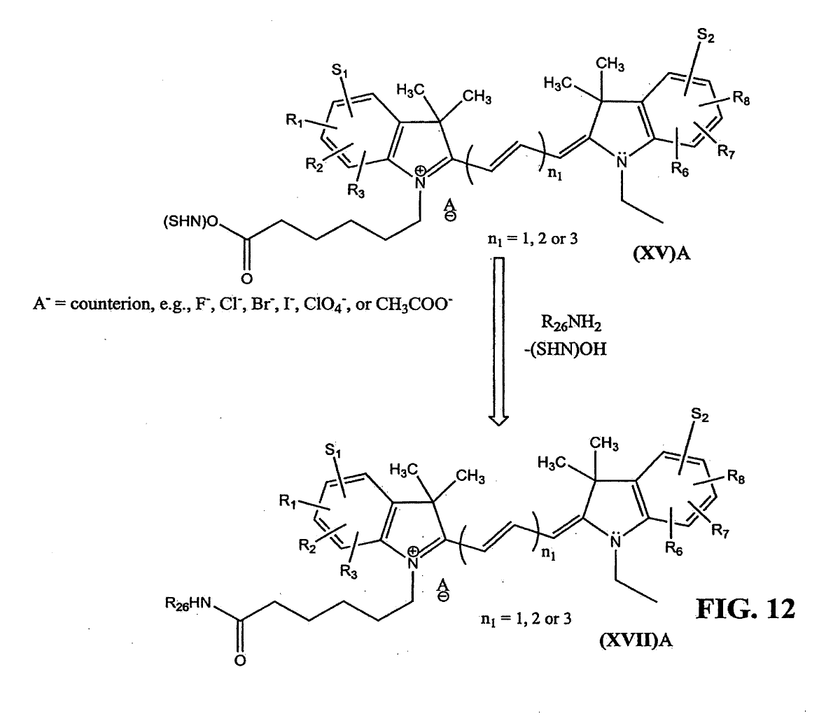

Any of the dyes described herein, e.g., dyes that include cations of Structures (1), (VIII) can be reacted with other compounds, e.g., oligomers or polymers that contain amine-, alcohol-, or thiol-groups, such as targeting, ligands (e.g., small molecule peptides, proteins, protein fragments, peptides, antibodies, carbohydrates, or antigens), to provide conjugates. For example, FIGS. 11 and 12 show, respectively, reaction of dyes of Structure (XV)A with hydroxyl-containing moieties, and amine-containing moieties.

In a typical conjugation procedure, all of the following steps can be performed under reduced light conditions in dimethyl sulfoxide (DMSO) at room temperature. In one procedure, each 50 μL reaction contains 20 mM triethylamine. (TEA), 1 mM of the desired ligand, and 1 mM of the desired dye. To effect the conjugation, the reaction mixture is constantly agitated for 18 hours in the dark. Additional general details for conjugation of dyes is discussed in Frangioni et al., Molecular Imaging, vol. 1(4), 354-364 (2002).

Specific proteins, protein fragments, peptides, antibodies, carbohydrates, or antigens that can be used to form the new conjugates are described, e.g., in Frangioni et al. in “MODIFIED PSMA LIGANDS AND USES RELATED THERETO”, WO 02/098885, filed on Feb. 7, 2002 (now issued as U.S. Pat. No. 6,875,886). A specific targeting ligand is the RGD peptide, which specifically binds to alphₐᵥβ₃ integrin. It is known that this integrin is overexpressed by various tumors, and thus, these RGD targeting peptides enable the dyes to preferentially label tumors that overexpress these integrins. Other targeting ligands include melanocyte stimulating hormone (MSH), which targets melanoma cells, or bombesin, somatostatin, or Sandostatin™ (synthetic), which target somatostatin receptors.

### Applications

The dyes and dye conjugates, e.g., dye-biomolecule conjugates, can be used for, e.g., optical tomographic, endoscopic, photoacoustic, and sonofluorescent applications for the detection, imaging, and treatment of tumors and other abnormalities.

The dyes and dye conjugates can also be used for localized therapy. This can be accomplished, e.g., by attaching a porphyrin or other photodynamic therapy agent conjugates to accumulate selectively in the target site; and shining light of an appropriate wavelength to activate the agent. Thus, the new conjugates can be used to detect, image, and treat a section of tissue, e.g., a tumor. In addition, the dyes and conjugates can be used for detecting the presence of tumors and other abnormalities by monitoring the blood clearance profile of the conjugates, for laser assisted guided surgery for the detection of small micrometastases of, e.g., somatostatin subtype 2 (SST-2) positive tumors, and for diagnosis of atherosclerotic plaques and blood clots.

### Dyes and Dye Conjugate Compositions

The dyes and dye conjugates can be formulated into diagnostic and therapeutic compositions for enteral, or parenteral administration. Generally, these compositions contain an effective amount of the dye or dye conjugate, along with conventional pharmaceutical carriers and excipients appropriate for the type of administration contemplated. For example, parenteral formulations include the dye or dye conjugate in a sterile aqueous solution or suspension. Parenteral compositions can be injected directly into a subject at a desired site, or mixed with a large volume parenteral composition for systemic administration. Such solutions can also contain pharmaceutically acceptable buffers and, optionally, electrolytes, such as sodium chloride.

Formulations for enteral administration, in general, can contain liquids, which include an effective amount of the desired dye, or dye conjugate in aqueous solution, or suspension. Such enteral compositions can optionally include buffers, surfactants, and thixotropic agents. Compositions for oral administration can also contain flavoring agents, and other ingredients for enhancing their organoleptic qualities.

Generally, the diagnostic compositions are administered in doses effective to achieve the desired signal strength to enable detection. Such doses can vary, depending upon the particular dye or dye conjugate employed, the organs or tissues to be imaged, and the imaging equipment being used. For example, Zeheer et al., Nature Biotechnology, 19, 1148-1154 (2001) uses 0.1 μmol/kg as a dose for IRDye78 conjugates in vivo. The diagnostic compositions can be administered to a patient systemically, or locally to the organ, or tissue to be imaged, and then the patient is subjected to the imaging procedure.

### Other Embodiments

A number of embodiments have been described. Nevertheless, it will be understood that various modifications may be made without departing from the spirit and scope of the invention. Other embodiments are within the scope of the following claims.

1. A compound comprising a cation of Structure (I): 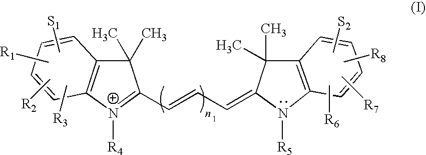
wherein
S₁, and S₂ are each independently a non-ionic oligomeric or polymeric solubilizing moiety;
n₁ is 1, 2 or 3;
R₁, R₂, R₃, R₆, R₇, and R₈ are each independently H, F, Cl, Br, I, C1-C6 straight-chain or branched alkyl, C1-C6 straight-chain or branched alkoxy, an aromatic ring having up to 6 carbon atoms, optionally substituted with one or more F, Cl, Br or I, or any two or more of R₁, R₂ and R₃ and/or any two or more of R₆, R₇ and R₈ may be bonded together to define a ring that includes between 5 and 12 carbon atoms, wherein the ring is optionally substituted with one or more F, Cl, Br, or I; and
R₄ and R₅ are each independently C1-C6 straight-chain or branched alkyl, an aromatic ring having up to 6 carbon atoms, optionally substituted with one or more F, Cl, Br or I, a non-ionic oligomeric or polymeric solubilizing moiety, or a moiety that includes at least one amine-, alcohol- or thiol-reactive carboxylic acid group, anhydride group, ester group, or isothiocyanate group.

2. A compound of claim 1, wherein the moiety that includes at least one amine-, alcohol- or thiol-reactive carboxylic acid group, anhydride group, ester group, or isothiocyanate group also includes a solubilizing moiety.

3. A compound of claim 1 comprising cations of Structure (I′)

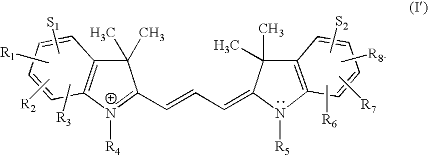

4. A compound of claim 1 comprising a cation of Structure (I″)

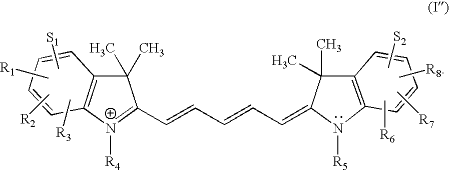

5. A compound of claim 1 comprising a cation of Structure (I′″)

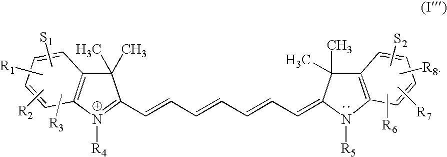

6. A compound of claim 1, wherein S₁ and S₂ are each selected such the compound has a solubility in 10 mM HEPES solution, pH 7.4, of greater than about 10 μM.

7. A compound of claim 1, wherein S₁ and S₂ are each independently selected from the group consisting of a polyethylene glycol, a polypropylene glycol, a copolymer of polyethylene oxide and propylene oxide, a carbohydrate, a dextran, and a polyacrylamide.

8. A compound of claim 1, wherein S₁ and S₂ are each independently of the form R₉(α)_{φ}, wherein φ is 0 or 1, α is O, S, CH₂, CH₂O, CO₂, or NR′ in which R′ is H or C1-C6 straight-chain or branched alkyl, and wherein R₉ is of the form (CH₂CH₂O)ₙ₃R″ in which R″ is H or C1-C6 straight-chain or branched alkyl, n₃ being an integer from 4 to 2,500.

9. A compound of claim 1, wherein R₁, R₂, R₃, R₆, R₇, and R₈ are each H.

10. A compound of claim 1, wherein R₄ is 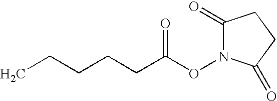
and wherein R₅ is C1-C6 alkyl.

11. A compound of claim 1, further comprising an anion selected from the group consisting of F⁻, Cl⁻, Br⁻, I⁻, ClO₄⁻, and CH₃COO⁻.

12. A reaction product of a compound of claim 1 and an amino- or hydroxyl- or thiol-containing moiety.

13. The reaction product of claim 12, wherein the amino-containing moiety is a small molecule peptide, protein, a polypeptide, an antibody, or an antigen.

14. The reaction product of claim 12, wherein the hydroxyl-containing group is a carbohydrate.

15. A compound of Structure (V): 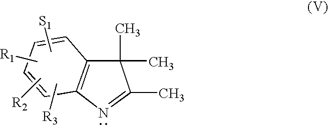
wherein
S₁ is a non-ionic oligomeric or polymeric solubilizing moiety; and
R₁, R₂, R₃ are each independently H, F, Cl, Br, I, C1-C6 straight-chain or branched alkyl, C1-C6 straight-chain or branched alkoxy, an aromatic ring having up to 6 carbon atoms, optionally substituted with one or more F, Cl, Br or I, or any two or more of R₁, R₂ and R₃ may be bonded together to define a ring that includes between 5 and 12 carbon atoms, wherein the ring is optionally substituted with one or more F, Cl, Br, or I.

16. A compound of claim 15, wherein S₁ is selected such that the compound of Structure (V) has a solubility in 10 mM HEPES solution, pH 7.4, of greater than about 10 μM.

17. A compound comprising a cation of Structure (VI) 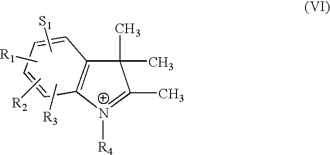
wherein
S₁ is a non-ionic oligomeric or polymeric solubilizing moiety;
R₁, R₂, R₃ are each independently H, F, Cl, Br, I, C1-C6 straight-chain or branched alkyl, C1-C6 straight-chain or branched alkoxy, an aromatic ring having up to 6 carbon atoms, optionally substituted with one or more F, Cl, Br or I, or any two or more of R₁, R₂ and R₃ may be bonded together to define a ring that includes between 5 and 12 carbon atoms, wherein the ring is optionally substituted with one or more F, Cl, Br, or I; and
R₄ is independently C1-C6 straight-chain or branched alkyl, an aromatic ring having up to 6 carbon atoms, optionally substituted with one or more F, Cl, Br or I, a non-ionic oligomeric or polymeric solubilizing moiety, or a moiety that includes at least one amine, alcohol- or thiol-reactive carboxylic acid group, anhydride group, ester group, or isothiocyanate group.

18. A compound of claim 17, wherein S₁ is selected such that the compound that comprises the cation of Structure (VI) has a solubility in 10 mM HEPES solution, pH 7.4, of greater than about 10 μM.

19. A compound of claim 17, further comprising an anion selected from the group consisting of F⁻, Cl⁻, Br⁻, I⁻, ClO₄⁻, and CH₃COO⁻.

20. A compound comprising a cation of Structure (VIII): 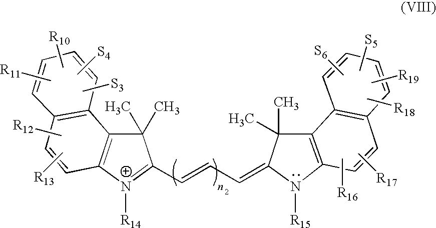
wherein
S₃, S₄, S₅ and S₆ are each independently a non-ionic oligomeric or polymeric solubilizing moiety;
n₂ is 1, 2 or 3;
R₁₀, R₁₁, R₁₂, R₁₃, R₁₆, R₁₇, R₁₈, and R₁₉ are each independently H, F, Cl, Br, I, C1-C6 straight-chain or branched alkyl, C1-C6 straight-chain or branched alkoxy, an aromatic ring having up to 6 carbon atoms, optionally substituted with one or more F, Cl, Br or I, or any two or more of R₁₀, R₁₁, R₁₂, and R₁₃; and/or any two or more of R₁₆, R₁₇, R₁₈, and R₁₉ may be bonded together to define a ring that includes between 5 and 12 carbon atoms, wherein the ring is optionally substituted with one or more F, Cl, Br, or I; and
R₁₄ and R₁₅ are each independently C1-C6 straight-chain or branched alkyl, an aromatic ring having up to 6 carbon atoms, optionally substituted with one or more F, Cl, Br or I, a non-ionic oligomeric or polymeric solubilizing moiety, or a moiety that includes at least one amine-, alcohol- or thiol-reactive carboxylic acid group, anhydride group, ester group, or isothiocyanate group.

21. A compound of claim 20, wherein the moiety that includes at least one amine-, alcohol- or thiol-reactive carboxylic acid group, anhydride group, ester group, or isothiocyanate group also includes a solubilizing moiety.

22. A compound of claim 20 comprising a cation of Structure (VIII′)

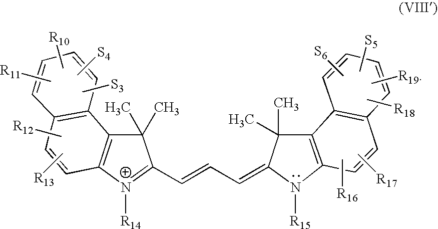

23. A compound of claim 20 comprising a cation of Structure (VIII″)

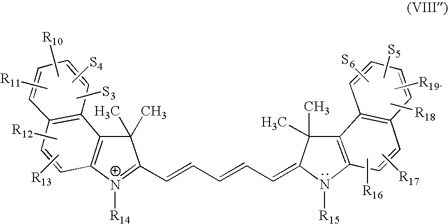

24. A compound of claim 20 comprising a cation of Structure (VIII′″)

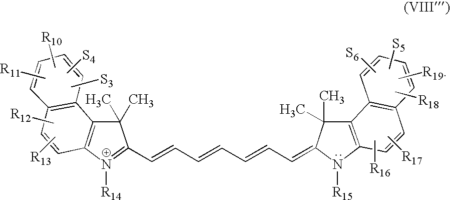

25. A compound of claim 20, wherein S₃, S₄, S₅ and S₆ are selected such the compound has a solubility in 10 mM HEPES solution, pH 7.4, of greater than about 10 μM.

26. A compound of claim 20, wherein S₃, S₄, S₅ and S₆ selected such that the compound has a solubility in 10 mM HEPES solution, pH 7.4, of greater than about 50 μM.

27. A compound of claim 20, wherein S₃, S₄, S₅ and S₆ are each independently selected from the group consisting of a polyethylene glycol, a polypropylene glycol, a copolymer of polyethylene oxide and propylene oxide, a carbohydrate, a dextran, and a polyacrylamide.

28. A compound of claim 20, wherein S₃, S₄, S₅ and S₆ are each independently of the form R₉(α)_{φ}, wherein φ is 0 or 1, α is O, S, CH₂, CH₂O, CO₂, or NR′ in which R′ is H or C1-C6 straight-chain or branched alkyl, and wherein R₉ is of the form (CH₂CH₂O)ₙ₃R″ in which R″ is H or C1-C6 straight-chain or branched alkyl, n₃ being an integer from 4 to 2,500.

29. A compound of claim 20, wherein R₁₀, R₁₁, R₁₂, R₁₃, R₁₆, R₁₇, R₁₈, and R₁₉ are each H.

30. A compound of claim 20, wherein R₄ is 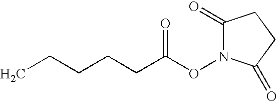
and wherein R₅ is C1-C6 alkyl.

31. A compound of claim 20, further comprising an anion selected from the group consisting of F⁻, Cl⁻, Br⁻, I⁻, ClO₄⁻, and CH₃COO⁻.

32. A reaction product of a compound of claim 20 and an amino- or hydroxyl- or thiol-containing moiety.

33. A compound of Structure (XII): 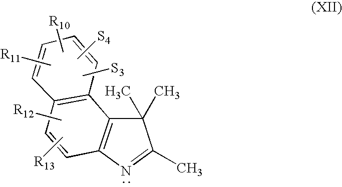
wherein
S₃, and S₄ are each independently a non-ionic oligomeric or polymeric solubilizing moiety; and
R₁₀, R₁₁, R₁₂, and R₁₃ are each independently H, F, Cl, Br, I, C1-C6 straight-chain or branched alkyl, C1-C6 straight-chain or branched alkoxy, an aromatic ring having up to 6 carbon atoms, optionally substituted with one or more F, Cl, Br or I, or any two or more of R₁₀, R₁₁, R₁₂, and R₁₃ may be bonded together to define a ring that includes between 5 and 12 carbon atoms, wherein the ring is optionally substituted with one or more F, Cl, Br, or I.

34. A compound of claim 33, wherein S₃, and S₄ are selected such that the compound of Structure (XII) have a solubility in 10 mM HEPES solution, pH 7.4, of greater than about 10 μM.

35. A compound comprising a cation of Structure (XII) 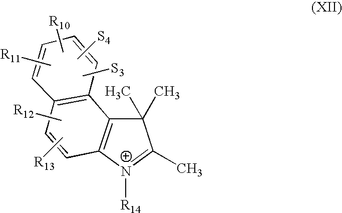
wherein
S₃, and S₄ are each independently a non-ionic oligomeric or polymeric solubilizing moiety;
R₁₀, R₁₁, R₁₂, and R₁₃ are each independently H, F, Cl, Br, I, C1-C6 straight-chain or branched alkyl, C1-C6 straight-chain or branched alkoxy, an aromatic ring having up to 6 carbon atoms, optionally substituted with one or more F, Cl, Br or I, or any two or more of R₁₀, R₁₁, R₁₂, and R₁₃ may be bonded together to define a ring that includes between 5 and 12 carbon atoms, wherein the ring is optionally substituted with one or more F, Cl, Br, or I; and
R₁₄ is C1-C6 straight-chain or branched alkyl, an aromatic ring having up to 6 carbon atoms, optionally substituted with one or more F, Cl, Br or I, a non-ionic oligomeric or polymeric solubilizing moiety, or a moiety that includes at least one amine-, alcohol- or thiol-reactive carboxylic acid group, anhydride group, ester group, or isothiocyanate group.

36. A compound of claim 35, wherein S₃, and S₄ are each selected such that compounds that comprise cations of Structure (XIII) have a solubility in 10 mM HEPES solution, pH 7.4, of greater than about 10 μM.

37. A compound of claim 35, further comprising an anion selected from the group consisting of F⁻, Cl⁻, Br⁻, I⁻, ClO₄⁻, and CH₃COO⁻.

38. A dye comprising:
a positively charged nitrogen-containing dye core comprising a conjugated tri-, penta-, or heptamethine system;
one or more non-ionic solubilizing molecular arms bonded to the dye core; and
optionally, one or more functionalizable molecular arms bonded to the dye core, wherein the one or more functionalizable molecular arms each comprise an amine-, alcohol-, or thiol-reactive carboxylic acid group, anhydride group, ester group, or isothiocyanate group.

39. A dye of claim 38, wherein the tri-, penta- or heptamethine is an unsubstituted system.

40. A dye of claim 38, wherein the positively charged nitrogen-containing dye core has a single positive charge.

41. A dye of claim 38, wherein the one or more solubilizing molecular arms are selected such that the dye has a solubility in 10 mM HEPES solution, pH 7.4, of greater than about 10 μM.

42. A dye of claim 38, wherein the one or more solubilizing arms are also functionalized with an amine-, alcohol-, or thiol-reactive carboxylic acid group, anhydride group, ester group, or isothiocyanate group.

43. A dye of claim 38, wherein the dye has a maximum excitation and/or a maximum emission, measured in 10 mM HEPES solution, pH 7.4, of from about 525 nm to about 875 nm.

44. A dye of claim 38, wherein the one or more non-ionic solubilizing molecular arms and/or the one or more functionalizable molecular arms are bonded to the tri-, penta-, or heptamethine system of the dye.

45. A dye of claim 38 comprising two or more functionalizable molecular arms.

46. A method of making a compound, the method comprising:
attaching a non-ionic solubilizing moiety to a functionalized aniline having a hydrogen atom ortho to an amino group of the aniline to provide an aniline having a solubilizing arm;
converting the aniline having the solubilizing arm to its corresponding hydrazine; and
cyclizing the hydrazine with a cyclizing moiety to form a nitrogen-containing, fused heterocyclic ring having points of fusion at a point of attachment of the hydrazine and ortho to the point of attachment of the hydrazine.

47. The method of claim 46, further comprising quaternizing a nitrogen atom of the nitrogen-containing fused heterocyclic ring with a quaternizing moiety to provide a quaternized nitrogen-containing compound.

48. The method of claim 47, wherein the quaternizing moiety comprises an amine-, alcohol-, or thiol-reactive group.

49. The method of claim 48, wherein the amine-, alcohol-, or thiol-reactive group is a carboxylic acid group, anhydride group, ester group, or isothiocyanate group.

50. The method of claim 47, further comprising coupling quaternized nitrogen-containing compounds with a coupling moiety to provide a nitrogen-containing core bearing a positive charge and comprising a conjugated tri-, penta, or heptamethine system bridging fused heterocyclic rings.

51. The method of claim 46, wherein the non-ionic solubilizing moiety is polymeric.

52. The method of claim 46, wherein the cyclizing moiety is methyl isopropyl ketone.

53. A method of making a conjugate, the method comprising:
providing a compound of claim 1, wherein the compound has at least one amine-, alcohol-, or thiol-reactive carboxylic acid group, anhydride group, ester group, or isothiocyanate group; and
reacting the provided compound with a moiety that includes an amino, hydroxyl, or thiol group to provide a conjugate.

54. A method of imaging or treating abnormal tissue and/or cells, the method comprising:
administering to a subject a conjugate comprising a reaction product of any compound of claim 1, and a amino- hydroxyl- or thiol-containing moiety, wherein the amino-, hydroxyl-, or thiol-containing moiety has binding sites complementary with to on the abnormal tissue and/or cells; and
irradiating the conjugate with radiation at a wavelength that the conjugate absorbs.

55. The method of claim 54, further comprising detecting and/or quantifying absorbed and/or emitted radiation.

56. The method of claim 53, wherein the amino- hydroxyl- or thiol-containing moiety is a small molecule peptide, protein, polypeptide, antibody, or an antigen.
